(19) United States
(12) Patent Application Publication
(10) Pub. No.: US 2021/0398645 A1
(43) Pub. Date: Dec. 23, 2021

(54) METHODS AND SYSTEMS FOR MEASURING DIET AND NUTRITION IN CHILDREN

(71) Applicant: NUtech Ventures, Lincoln, NE (US)

(72) Inventors: Dipti Dev, Lincoln, NE (US); Santosh K. Pitla, Lincoln, NE (US); Ashu Guru, Lincoln, NE (US); Hunter Guru, Lincoln, NE (US); Anthony Weng, Lincoln, NE (US)

(21) Appl. No.: 17/287,618

(22) PCT Filed: Oct. 24, 2019
(86) PCT No.: PCT/US19/57770

§ 371 Date: Apr. 22, 2021

Related U.S. Application Data
(60) Provisional application No. 62/749,700, filed on Oct. 24, 2018.

(51) Int. Cl. G16H 20/60; G01G 19/40; G16H 10/20; G16H 40/67

(52) U.S. Cl. CPC G16H 20/60; G16H 40/67; G16H 10/20; G01G 19/40

(57) ABSTRACT

A tray assembly for monitoring food consumed from one or more dishes supported by the tray assembly includes a housing defining an interior and having an upper surface for supporting the one or more dishes. One or more weight assemblies are disposed in the interior of the housing. Each weight assembly includes a weight sensor configured to detect a weight of one dish of the one or more dishes and to provide a weight signal corresponding to the detected weight of the one dish. A difference between the weight taken at a first time during a meal and at a second time, after the first time, during the meal being generally indicative of an amount of food consumed during the meal from the one or more dishes.

### CROSS-REFERENCE TO RELATED APPLICATIONS

This application claims priority to U.S. Provisional Application No. 62/749,700, filed Oct. 24, 2018, the entirety of which is hereby incorporated by reference.

### STATEMENT OF GOVERNMENT SUPPORT

This invention was made with government support under 2016-31100-06031, 2016-31200-06031, NI17HMFPXXXXG026, NI17HFPXXXXXG047, NI18HMFPXXXXG022, NI18HFPXXXXXG045, NI19HFPXXXXXG019 and NI19HMFPXXXXG032 awarded by the United States Department of Agriculture, National Institute of Food and Agriculture. The government has certain rights in the invention.

### FIELD

The present disclosure relates to smart kitchenware and, more particularly, to a tray assembly that may be used to monitor food intake.

### BACKGROUND

Excessive body weight is the leading nutrition-related problem around the world. Many Americans, for example, are obese and/or suffer from weight-related health issues. Even more, childhood obesity in the United States is rising at an alarming rate. To address these issues, national policies, researchers, educators, and pediatricians conduct nutrition-related programs with children to encourage children to eat healthy foods (e.g., lean meats, fruits, vegetables, whole grain and low-fat dairy). However, at least known methods and systems for measuring food intake in children (e.g., to document the impact of nutrition-related policies and programs) are cumbersome, awkward, or cost-prohibitive to implement or use on a consistent basis.

### SUMMARY

In one aspect, a tray assembly is provided. The tray assembly supports dishes that hold various food items, and includes weight sensors that detect a weight of each dish and the food contained therein. Generally, a difference between weights taken at the beginning of a meal and at the end of the meal is indicative of an amount of food consumed during the meal. The tray assembly interfaces with a database to determine nutritional values of the food consumed. To account for the consumption of a variety of foods, each dish is individually weighed. The tray assembly is also configured to account for a spillage or sharing of food. A mobile “app” may be used to track and view detailed caloric and nutritional intake over time and/or compare with others' to encourage certain behaviors.

In another aspect, a tray assembly for monitoring food consumed from one or more dishes supported by the tray assembly includes a housing defining an interior and having an upper surface for supporting the one or more dishes. One or more weight assemblies are disposed in the interior of the housing. Each weight assembly includes a weight sensor configured to detect a weight of one dish of the one or more dishes and to provide a weight signal corresponding to the detected weight of the one dish. A difference between the weight taken a first time during a meal and at a second time, after the first time, during the meal being indicative of an amount of food consumed during the meal from the one or more dishes.

In another aspect, a method for monitoring food consumed from one or more dishes comprises measuring a weight of each dish and food contained therein at a first time during the meal using a tray assembly; measuring a weight of each dish and any of the food contained therein at a second time during the meal using the tray assembly; and determining the amount of food consumed by taking the difference between the weight taken at the first and second times.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF THE DRAWINGS

Features, aspects, and advantages of the present disclosure will become better understood when the following Detailed Description is read with reference to the accompanying drawings in which like reference characters represent like elements throughout, wherein:

FIG. 1 is a perspective of a tray assembly according to one embodiment of the present disclosure, with a plurality of dishes supported thereon;

FIG. 2 is a perspective of the tray assembly, with a top cover removed to show interior components of the tray assembly;

FIG. 3 is a side view of a weight assembly of the tray assembly;

FIG. 4 is a food consumption graph showing the weight of the food in a dish on the tray assembly versus time during a meal;

FIG. 5 is a screenshot of a menu home screen on a graphical user interface;

FIG. 6 is a screenshot of an add menu screen on the graphical user interface of FIG. 5;

FIG. 7 is a screenshot of the menu home screen of FIG. 5, after an additional menu has been created;

FIG. 8 is a screenshot of a children home screen on a graphical user interface;

FIG. 9 is a screenshot of a meal history screen on the graphical user interface of FIG. 8;

FIG. 10 is a screenshot of a meal detail screen on the graphical user interface of FIG. 8; and

FIG. 11 is a screenshot of the meal detail screen of FIG. 10, showing additional features.

Corresponding parts are indicated by corresponding reference characters throughout the several views of the drawings.

Although specific features of various examples may be shown in some drawings and not in others, this is for convenience only. Any feature of any drawing may be referenced and/or claimed in combination with any feature of any other drawing.

### DETAILED DESCRIPTION

The present disclosure relates to smart kitchenware and, more particularly, to a tray assembly that may be used to monitor food intake. The tray assembly monitors food consumed from one or more (e.g., a plurality of) dishes supported by the tray assembly. Examples described herein include a tray assembly with weight sensors that detect a weight of the dish of food. A difference between weights taken between two times during the meal (e.g., at the beginning of a meal and at the end of the meal), for example, may indicate an amount of food consumed during the meal from that dish. A user interface may be used to track caloric and nutritional intake over time.

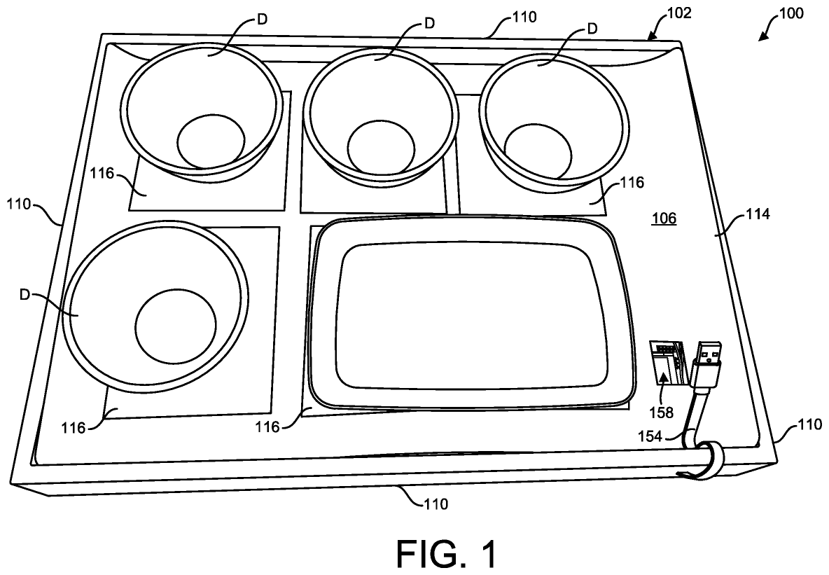

Referring to FIGS. 1-3, a tray assembly 100 according to one embodiment of the present disclosure is generally indicated at 100. The tray assembly 100 includes a housing 102 defining an interior 104 and having an upper surface 106 for supporting one or more dishes D containing food (not shown) thereon. The housing 102 includes a base 108 with side walls 110 extending upward therefrom. Two opposing side walls 110 include handles. In the illustrated embodiment, each handle is an opening 112 in the side walls 110 sized and shaped to receive a portion of the user's hand (e.g., fingers). The housing 102 includes a top cover or mat 114 defining the upper surface of the housing. Preferably, the mat 114 is selectively removable from the rest of the housing 102 (e.g., base 108 and side walls 110). Removing the mat 114 permits access to the interior 104 to clean the housing (if food is spilled) or access one or more components therein. For reasons that will become apparent, the mat 114 is flexible (e.g., the mat is a flexible mat). In one embodiment, the mat 114 is made of a food-grade material, such as silicon, although other suitable materials are within the scope of the present disclosure. The mat 114 may include dish placement indicators 116, such as geometric shapes, images, designs, number, letter, etc., that designate where the dishes D are to be placed on the upper surface 106 of the housing 102. Broadly, the dish placement indicator 116 can be anything that provides a visual indication of where the dishes D are to be placed on the mat 114. For example, the dish placement indicator 116 can have a color different than the color of the rest of the upper surface 106.

Each dish D is sized and shaped to hold various food items to be consumed during the meal. The dishes D can be the same size and shape or different sizes and shapes. In the illustrated embodiment, there are four small dishes D and one larger dish supported by the tray assembly 100, although other configurations are within the scope of the present disclosure. The larger dish D is generally suitable for holding an entrée (e.g., meat, fish, etc.) and the smaller dishes are generally suitable for holding side dishes (e.g., grains, vegetables, fruits, desert, etc.). The dishes D may be sized to hold amounts of food that are consistent with dietary recommendations (e.g., portion sizes appropriate for children). Alternatively, the tray assembly 100 may support any number of dishes D in various configurations. The mat 114 and/or dishes D may include or be fabricated from a dishwasher-safe material.

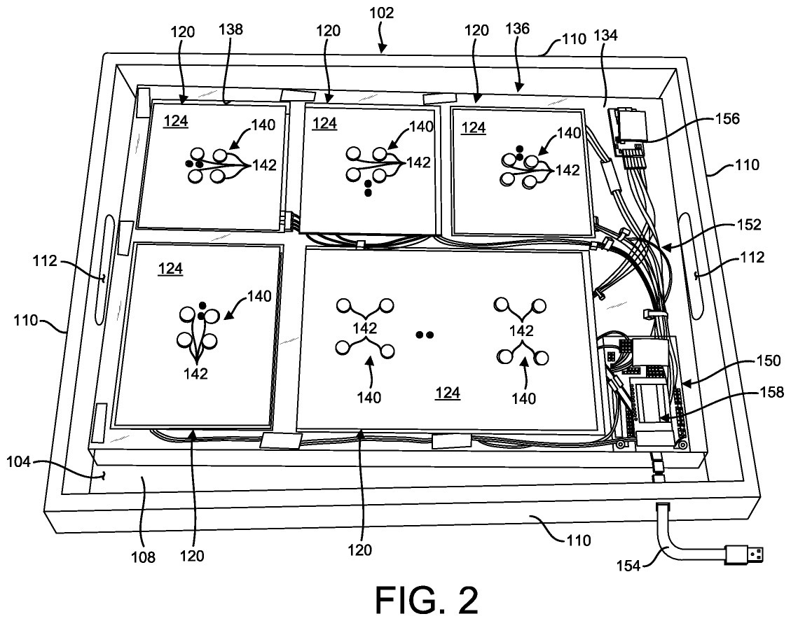

FIG. 2 shows the mat 114 removed or spaced from the rest of the housing 102. As shown, the interior 104 of the housing is configured (e.g., sized and shaped) to retain or house one or more components. The tray assembly 100 includes one or more (e.g., a plurality of) load or weight assemblies 120 disposed in the interior of the housing 102. Each weight assembly 120 is configured to measure the weight of one of the dishes D. Alternatively, the weight assemblies 120 may be oriented or arranged to detect any weight. For example, two weight assemblies 120 may be oriented and arranged to detect the weight of one dish D. In the illustrated embodiment, the tray assembly 100 includes five weight assemblies 120, although more or fewer weight assemblies are within the scope of the present disclosure. For example, the tray assembly 100 can include 1, 2, 3, 4, 6, 7, 8, 9, 10 or more weight assemblies 120. Each weight assembly 120 is configured to detect weights at various areas of the tray assembly 100. The weight assemblies 120 enable food consumption to be tracked in a non-invasive, non-distracting manner. For example, a difference between weights taken at the beginning of a meal and at the end of the meal is generally indicative of an amount of food consumed during the meal from the one or more dishes D.

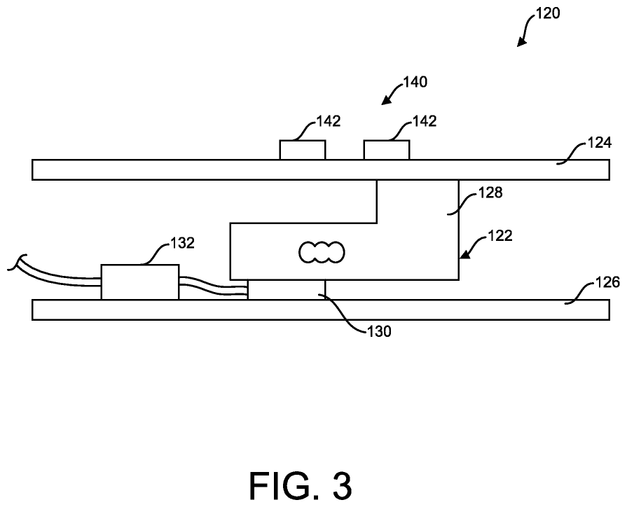

Referring to FIG. 3, each weight assembly 120 includes at least one weight sensor 122 configured to detect the weight of one of the dishes D and any food contained therein. As used herein, the weight detected by the weight assembly may include both the weight of the dish D and any food contained (e.g., held) by the dish. For example, the detected weight at a first time during the meal (e.g., the beginning of the meal) may include both the weight of the dish D and the food held by the dish, while the detected weight at the end of the meal may include only the weight of the dish because all the food was eaten. The weight sensor 122 is configured to generate or provide a weight signal corresponding to the detected weight of the one dish D. One example of a suitable weight sensor 122 is a load cell, although the use of other types of weight sensors are within the scope of the present disclosure. Each weight assembly 120 further includes an upper plate 124 and a lower plate 126. The weight sensor 122 is disposed between the upper and lower plates 124, 126. In the illustrated embodiment, each weight sensor 122 is a load cell that includes a bracket 128 fixed to the underside of the upper plate at one end and a series of strain gauges 130 (e.g., at least one strain gauge) at the other end. The bracket 128 is generally L-shaped and imparts a moment or rotational force on the strain gauges 130 due to the weight of a dish D placed over the weight assembly 120. The bracket 128 also maximizes the area (e.g., upper plate 124 supporting the dish D) in contact between the strain gauges 130 and the upper plate 124 to provide a more accurate measurement. The strain gauges 130 interconnect the bracket 128 to the lower plate 126. In this embodiment, the strain gauges 130 provide the weight signal. An amplifier 132 may be included to boost the power of the weight signal generated by the weight sensor 122 (e.g., strain gauges). This boost of power to the voltages corresponding to the ranges of the weight signal enables smaller changes in the weight to be detected. The upper and lower plates 124, 126 provide greater stability to a dish D supported by the weight assembly 120 than single point supports.

The weight assemblies 120 are configured to detect weights at various areas of the tray assembly 100. As shown in FIG. 2, there are five weight assemblies 120 spaced apart over the housing 102. The weight assemblies 120 rest on and are secured to the base 108 of the housing 102 (e.g., the lower plate 126 is secured to the base). The upper plates 124 of the weight assemblies 120 are generally co-planar with one another and define (e.g., partially define) a mat support surface 134 the mat 114 rests on. The tray assembly 100 also includes an interior housing or cover 136 which is configured to generally cover and protect the weight assemblies 120 and other components, as described herein, within the housing 102. In the illustrated embodiment, the interior cover 136 is shown generally transparent to show the component housed therein. The interior cover 136 protects the components from food spills that may occur during the meal. The interior cover 136 also defines a portion of the mat support surface 134. The interior cover 136 defines weight assembly openings 138 sized and shaped to receive the upper plates 124 of the weight assemblies 120. The mat 114 rests on (e.g., is supported by) the mat support surface 134. Thus, each weight assembly 120 is disposed below the mat 114. As a result, each weight assembly 120 supports at least a portion of the mat 114. Due to the flexibility of the mat 114 (e.g., the lack of rigidity), the mat 114 is configured to transfer the weight of the one or more dishes D, placed thereon, to the one or more weight assemblies 120. Specifically, the mat 114 is configured to transfer the weight of the dish D to the weight assembly 120 the dish is placed over.

Referring to FIGS. 2 and 3, each weight assembly 120 may also include a dish locator 140 (broadly, at least one dish locator) configured to position one dish D of the one or more dishes over the weight assembly when the dish is supported by the tray assembly 100. In the illustrated embodiment, each dish locator 140 is one or more magnets 142, although other configurations are within the scope of the present disclosure. If more than one magnet 142 is used, the magnets are grouped together, as illustrated. The magnets 142 are configured to interact (e.g., attract) a dish magnet (not shown) on each dish D to position the dish over the weight assembly 120. As a result, the dishes D generally snap into place when placed on the tray assembly 100. The dish locator 140 and dish placement indicator 116 preferably work together to help a child or food service professional properly position the dishes D on the tray assembly 100 to ensure the weight of each dish is properly measured. The dish magnet may be embedded in the dish D, secured to the bottom of the dish, or a sticky magnetic material adhered to the bottom of the dish. Alternatively, the dish D may be made out of a magnetic material. Where a weight assembly 120 only includes one dish locator 140 (e.g., one group of magnets), the dish locator is generally centered on the weight assembly 120, specifically on the upper plate 124. Where a weight assembly 120 includes two or more dish locators 140, the dish locators are generally spread out evenly on the weight assembly 120. A weight assembly 120 will typically include more than one dish locator if the dish D the weight assembly supports and measures is large (e.g., an entree size dish).

Referring to FIG. 2, the tray assembly 100 includes a controller 150 (broadly, a computer) configured to receive the detected weights from the weight assemblies 120. Specifically, the controller 150 (e.g., tray assembly controller) is configured to receive the weight signal from each weight assembly 120. The controller 150 may also receive signals from other sensors and/or buttons (not shown) that may be included with the tray assembly 100. The controller 150 is disposed in the interior 104 of the housing 102. The controller 150 is communicatively coupled (either wired or wirelessly) to the weight assemblies 120. In the illustrated embodiment, a series of wires 152 communicatively couples the weight assemblies 120 to the controller 150. The controller 150 includes a CPU or processor (e.g., a tray assembly processor) and RAM or memory (broadly, non-transitory computer-readable storage medium). The controller 150 provides the computing engine that drives the operation of the tray assembly 100, as will be described in more detail below. Broadly, the memory includes (e.g., stores) processor-executable instructions for controlling the operation of the processor. The instructions embody one or more of the functional aspects of the tray assembly 100, with the processor executing the instructions to perform said one or more functional aspects. The controller 150 also includes a wireless communications port (e.g., a wireless transmitter), such as a Bluetooth port and/or a Wi-Fi port so the controller can wirelessly communicate with other devices. An external power cord 154 (e.g., charging cord) is connected to the controller 150 to power the tray assembly 100. In the illustrated embodiment, the power cord 154 includes a USB connector configured to connect to an external power source such as a battery. Alternatively, the tray assembly 100 may include a battery (e.g., rechargeable battery).

The controller 150 is coupled to the weight assemblies 120 to receive and identify the one or more detected weights associated with the one or more dishes D. These weights are then used to determine an amount of food consumed during the meal from the dishes D. Each weight is associated with the food held in the dish D. Once associated with a type of food, the weights and food types can then be used to determine the nutritional values of the food consumed by referencing a nutritional database, such as the United States Department of Agriculture's (USDA) database. Broadly, the weight of the food in each dish D interfaces with USDA nutrition database to determine the nutrition values of the foods consumed. These nutritional values, along with other information such as type of foods consumed, caloric intake, etc., can then be presented to interested parties such as parents, school administrators, etc. and/or recorded and tracked over time, as explained in more detail below.

In one embodiment, the controller 150 is communicatively coupled to a server (not shown) hosting a database (e.g., food consumption database) which records and stores the weights detected by the weight assemblies 120. The controller 150 can then send a controller signal to the server corresponding to the detected weights of the one or more dishes D. The server receives the controller signal and records the detected weights of the one or more dishes D in the database, to be later used by the server. For example, the server can determine (e.g., is configured to determine) the amount of food consumed for each dish D (e.g., food type) by taking the difference between the weight taken at a first time during the meal and at a second time, after the first time, during the meal, after receiving the controller signal (e.g., based on the controller signal). For example, the first time may correspond to the beginning of the meal or any time thereafter and the second time may correspond to the end of the meal or anytime thereafter. Further, more than two times (e.g., first, second, third, fourth, etc. times) may be used to determine the amount of food consumed, as described below. The server can also be linked to and reference the nutritional database. This way, the server can determine (e.g., is configured to determine) the nutritional value of the food consumed based on referencing the amount of food consumed with the nutritional value of the food consumed. Alternatively, the controller 150 can determine (e.g., is configured to determine) the amount of food consumed for each dish D (e.g., food type) by taking the difference between the weight taken at a first time during the meal and at a second time, after the first time, during the meal, after receiving the weight signals (e.g., based on the weight signals). For example, the first time may correspond to the beginning of the meal or any time thereafter and the second time may correspond to the end of the meal or anytime thereafter. Further, more than two times (e.g., first, second, third, fourth, etc. times) may be used to determine the amount of food consumed, as described below. The controller 150 can then send this information (e.g., amount of food consumed) to the server via the controller signal. It is understood the server may be communicatively coupled to many (e.g., hundreds or thousands of) tray assemblies 100. In some embodiments, the controller 150 may possess some or all of the capabilities of the server described herein.

The controller 150 may be communicatively coupled to the server by a wireless network, such as Wi-Fi. The wireless network may be a local area network for the meal location (e.g., the Wi-Fi at a school or house). Preferably, the controller 150 stores instructions for automatically establishing a connection with the server over the local area network. The controller 150 is programmed to work toward full connectivity with the server, maintain that connectivity after it is established, and re-establish such connectivity if a break subsequently occurs. The ability for the controller 150 to automatically connect to the server over the local area network, simplifies and streamlines the data collection process and prevents an operator or child from having to connect the controller (broadly, the tray assembly 100) to the server before a meal. For example, the controller 150 includes instructions for automatically connecting to the server when the tray assembly 100 is within the range of the local area network, such as when the tray assembly 100 is in a lunch room of a school. Preferably, the controller 150 is continuously sending the controller signal (e.g., the detected weights) to the server while the controller is on and connected to the wireless network. If the controller 150 is unable to connect or disconnected from the local area network, the controller is configured to store detected weights (e.g., collected data), either on the controller or via the storage in the external device port 156 (described below), and then send them to the server once the controller is connected to the local area network.

The controller 150 preferably includes a display 158 to provide an indication of when the controller 150 is communicatively coupled to the local area network. For example, the display 158 can be a light (e.g., light emitting diode (LED) light) that turns on/off, flashes and/or changes color based on whether the controller 150 is on/off and/or connected to the local area network. In one embodiment, the light is not illuminated when the controller 150 (e.g., tray assembly 100) is turned off, illuminated with one color (e.g., red) when the controller is on but not connected to the local area network, illuminated and flashing when the controller is on and establishing a connection with the local area network, and illuminated with a second color (e.g., green) when the controller is on and connected to the local area network. Other configurations are within the scope of the present disclosure. For example, the display 158 can be used as a menu screen, an indicator to show errors and/or a communicative tool for user to interact with in general. The tray assembly 100 may include a switch (not shown) to turn the controller 150 on and off or the controller can turn on automatically upon being connected to a power source. Once the controller 150 is turned on, the weight assemblies 120 automatically start measuring the weight thereon. The weight assemblies 120 continuously monitor the weight until the controller 150 is turned off.

In the illustrated embodiment, the controller 150 includes an external device port 156 which allows an external device, such as a storage device, to be coupled to the controller. This allows the controller 150 to receive information from an external source. For example, in one embodiment, the external device port 156 is configured to receive a storage device, such as a flash drive or SD card, containing configuration settings (e g, name, password) of the wireless network the controller 150 is to connect to in order to communicate with the server. In this example, a computer, such as a laptop or desktop computer, may be used to load the configuration settings onto the storage device and then the storage device is inserted into the external device port 156 to load the configuration setting onto the controller 150, providing the controller with the necessary information to connect to the wireless network. It is appreciated other types of information, such as software updates, may also be loaded onto the controller 150 by this method. Alternatively, the external device port 156 can be used to receive an external storage device to store the data (e.g., measured weights) and later loaded into the database, to permit the data to be collected even without a wireless connection.

The controller 150 and/or server are configured to determine when food in the dishes D have been spilled or otherwise not eaten but removed from the dish D, such as when food is shared with another. This way the uneaten food (e.g., weight) is not included in the amount of food (e.g., weight) consumed by the child. To determine when food has been spilled, for example, the weight of the dish D holding the food is analyzed over time. Broadly speaking, if the weight of the dish D drops significantly in a relatively short period of time, the controller 150 and/or server determines, by analyzing the weights and times in the database, that that weight of food has not been eaten and excludes the weight of the uneaten food from the total amount of food consumed. Similarly, by identifying a sudden increase in the weight, the controller 150 and/or server can determine that an additional portion of food has been given to the child.

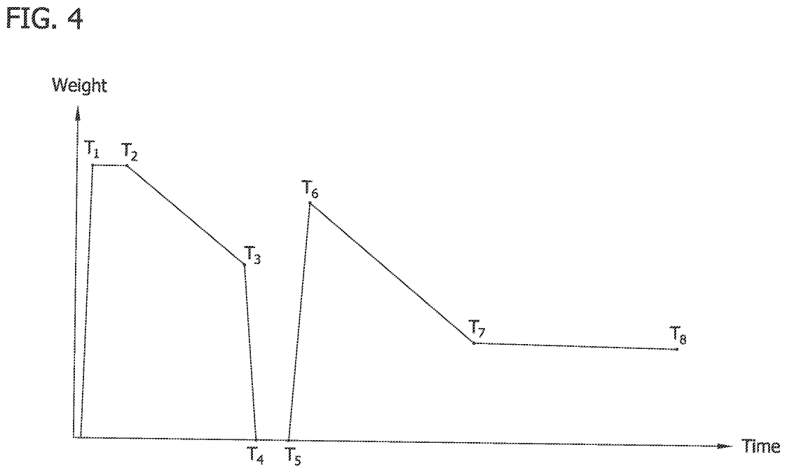

FIG. 4 is an exemplary food consumption graph showing the weight of the food for a particular dish D on the tray assembly 100 versus time for a meal to further illustrate this process, however, it is understood the controller 150 and/or server can create and use this graph as well. At T₁ the dish D holding the food is placed on the tray assembly 100. Accordingly, a relatively large weight is recorded, as shown. At T₂ the child starts eating the food from the dish D and, as a result, the weight of the dish gradually reduces. At T₃ the dish D is knocked off the tray assembly 100 resulting in a sudden and complete drop in the weight sensed by the weight assembly 120, as indicated (e.g., the weight sensed is zero). At T₅, the dish D is refilled with food and placed back on the tray assembly 100, resulting in a sudden and large increase in the weight. At T₆ the child continues to eat the food from the dish D. At T₇ the child has finished eating the food in the dish D and/or the dish is empty. At T₈ the meal ends. Using this information, the controller 150 and/or server can take the periods where the food was being consumed (e.g., when the weight was gradually being reduced) to determine the amount of the food consumed by the child. This is done by taking the difference between the local maximums and minimums (e.g., first and second times, respectively). In this case, the amount (e.g., weight) of food consumed is equal to T₂-T₃ plus T₆-T₇ (e.g., the difference between first and second times plus the difference between third and fourth times). Because decrease in weight between T₃ and T₄ was sudden, the controller 150 and/or server determines that this food was not eaten and excludes this difference from the amount of food calculation. Sudden may be considered 10 seconds or less, or more preferably 8 seconds or less, or more preferably 6 seconds or less, or more preferably, 5 seconds or less, or more preferably, 4 seconds or less, or more preferably 3 seconds or less, or more preferably 2 seconds or less, or more preferably 1 second or less. Similarly, because an increase in the weight between T₅ and T₆ was also sudden, the controller 150 and/or server can also determine that a second portion of food was given to the child. In this embodiment, if the dish D was only returned to the tray assembly but not filled with food (or returned with food but not eaten), the weight detected would not change after the dish was returned, indicating no additional food was provided (or no food was eaten). Thus, the controller 150 and/or server can determine when food has been spilled (or otherwise uneaten but removed from the dish D) and/or added to a dish by analyzing the weight sensed by the weight assembly 120 over time. Alternatively or conjunctively with analyzing the local maximum and minimums, the amount of food consumed can be determined using a time-series based algorithms (e.g., time-series filtering algorithms) such as a moving average filter. For example, the time-series based algorithm can be used for smoothing out irregularities in the detected weights (e.g., sensor noise) and/or account for the effects of sharing and/or spilling food, as described herein, and any other practices that would help to more accurately capture nutritional intake. The controller 150 and/or server can use the time-series based algorithms to determine the amount of food consumed.

In order to match the weights of the food consumed, as measured by the tray assembly 100, with the type of food consumed, the server needs to know what types of food are being served at that meal. In one embodiment, the types of food served at each meal are entered by an operator, such as a school administrator or childcare administrator, and stored in the database. For example, the server can host or be linked to a website where an operator can log on via a computer (broadly, a graphical user interface), such as a laptop or desktop computer, to enter the meal information into the database.

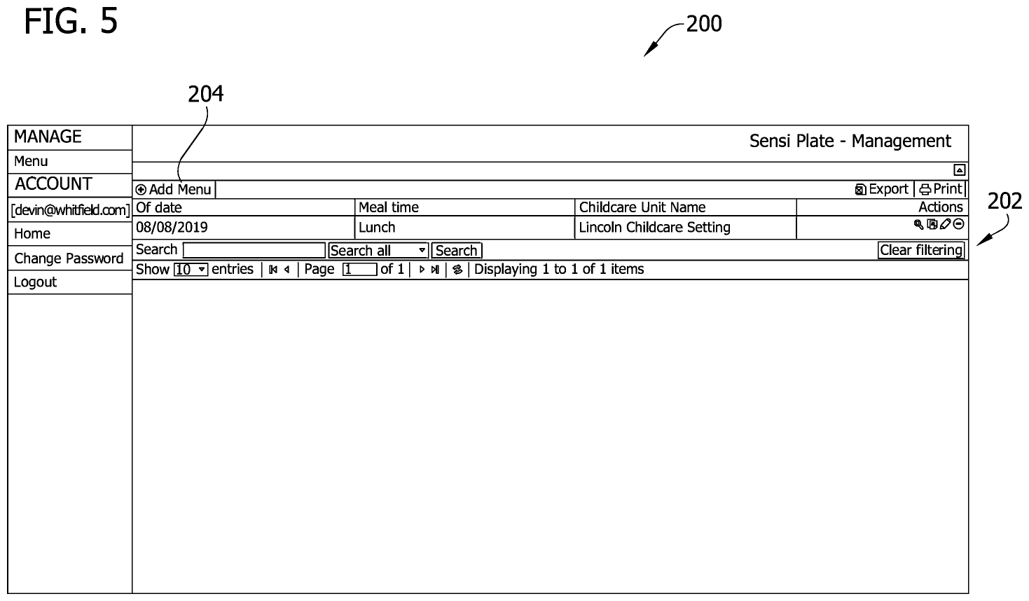

Referring to FIGS. 5-7, screenshots of example views (graphical interface) that may be displayed on the graphical user interface (e.g., computer) are shown. These screenshots are of webpages of a tray assembly management website and demonstrate how an operator can add food types or menus for each meal. The tray assembly management website is linked to the server and the database stored thereon. FIG. 5 is a screenshot of a menu home screen 200 showing the meals already entered into the database by the operator. The menu home screen 200 includes a meal table 202 listing, for each meal, the date of the meal was (or is to be) served, the type of meal (e.g., breakfast, lunch, diner, snack), and the name of the organization or unit serving (or associated with) the meal.

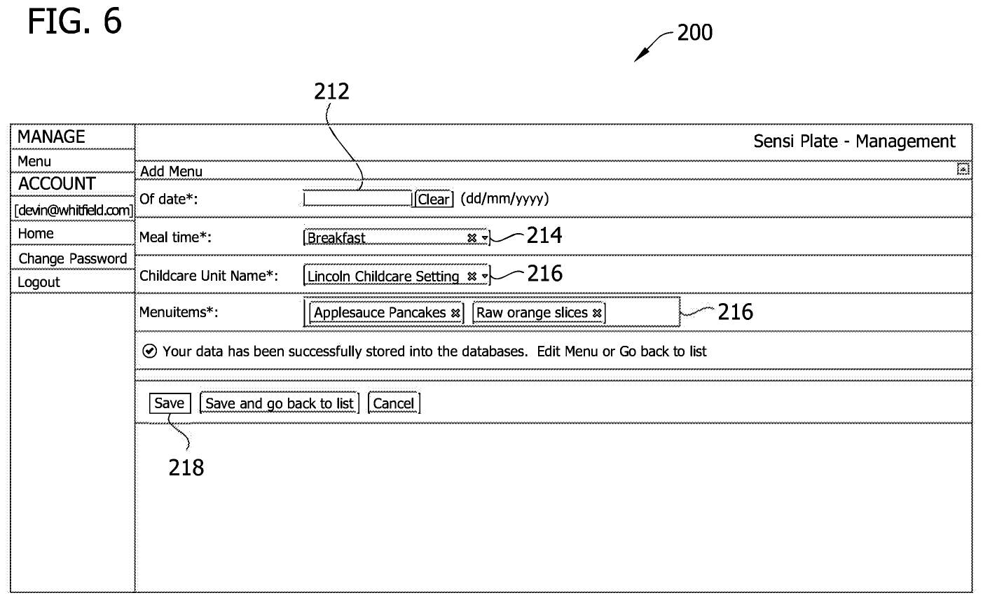

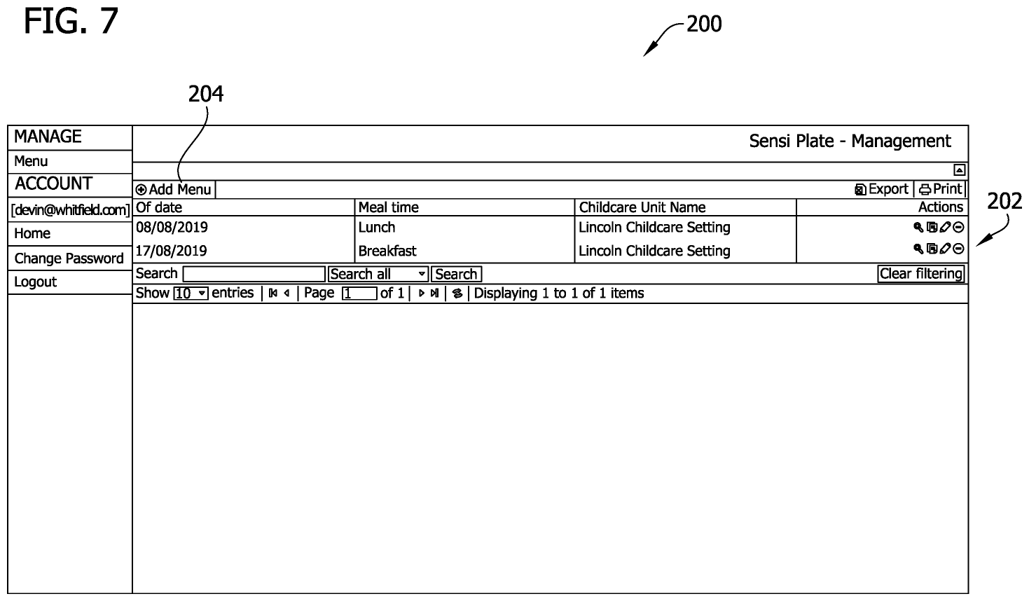

The menu home screen 200 includes an add menu button 204 which allows the operator to add a new meal or menu to the database. When the operator clicks on the add menu button 204, the operator is taken to the add menu screen 210 shown in FIG. 6. The add menu screen 210 includes a date field 212 where the operator can enter the date the meal was, is or will be served on, a meal time or type of meal field 214 for entering the type of meal, an organization or unit field for entering the organization serving or associated with the meal, and a food item field 216 for entering the different food items (e.g., types of food) served with the meal. Preferably, the food item field 216 is linked to a food item database so that only food items listed in the food item database can be entered into the food item field. This standardizes the way the types of food are entered into the database which simplifies associating the type of food with its nutritional value from the nutritional database. Accordingly, preferably the list of foods in the food item database is the same as the list of foods in the nutritional database, to allow for the quick retrieval of the nutritional values of the types of food. Once all the fields have been filled in by the operator, the operator presses the save button 218 to add the menu to the database. Pressing the save button 218 automatically takes the user back to the menu home screen 200, where the newly added meal is shown in the meal table 202 (FIG. 7). As explained above, once the food types of each meal are in the database, the nutritional value of the food consumed can be determined.

It is appreciated that other functions may be performed using the tray assembly management website. For example, the tray assembly management website can be used to allow operators to view and/or analyze the nutritional data on a daily basis, a per meal basis and/or a per child basis (or some combination thereof). The information (broadly, dietary intake data which includes the recorded weights) within the database may be used to identify, measure, and analyze consumer (e.g., child) eating behavior and measure the impact of interventions, such as by monitoring food intake. The dietary intake data may be shared with other users (e.g., for peer modeling), in addition to providing feedback based on a consumer's own data. Children are more likely to be influenced by peer models (e.g. their friends, cartoon characters). This may be used, for example, to encourage a child to try new foods (or target foods) that peers are eating. The tray assembly 100 may be used by a variety of different users (e.g., adults, children, clinical settings, childcare settings) and in a variety of different settings.

In another embodiment, the tray assembly 100 may include a food identification system (not shown) configured to determine or identify (or facilitate the determination or identification of) the types of food being consumed (e.g., the food in the dishes D). The food identification system is communicatively coupled (either wired or wirelessly) to the controller 150 which can then send the information received from the food identification system to the server. The information provided by the food identification system, via one or more signals, can be the names of the types of food or identification information to be used by the controller 150 and/or server to identify the types of food (e.g., the controller and/or server are configured to identify the types of food in the dishes D based on the identification information from the food identification system). The food identification system can include one or more sensors such as temperature sensors, resistivity sensors, color sensors, mass sensors, and/or cameras (which can be used for, among other things, volume to estimate density). The use of other sensors to detect other characteristics of the food is within the scope of the present disclosure. For example, food identification system can include one or more cameras can take photos of the food, which are uploaded to the server which identifies the food. In another example, the food identification system includes one or more cameras that takes photos of the food which are used by controller 150 to identify the food. The controller 150 then sends the food identities to the server. One or more of the sensors used in the food identification system may be embedded into the dishes D, the weight assemblies 120 (e.g., upper plate 124), the housing 102, and/or the mat 114 (e.g., surface 106). In one embodiment, one or more cameras may be included in a retractable handle system (not shown), which capture a top-down view (e.g., plan view) of the tray assembly 100 when the handles of the retractable handle system are fully extended. These one or more cameras may be embedded in the retractable handles. Further in an effort to minimize the number of sensors needed to adequately identify the food, different combinations and/or numbers of sensors can be analyzed to determine which combination of fewest sensors provides the necessary information to adequately identify the type of food in the dish D. Moreover, the food identification system can be used with the manually entered food types, as described above, to confirm the types of food associated with the meal in the database as the types of food being served and consumed.

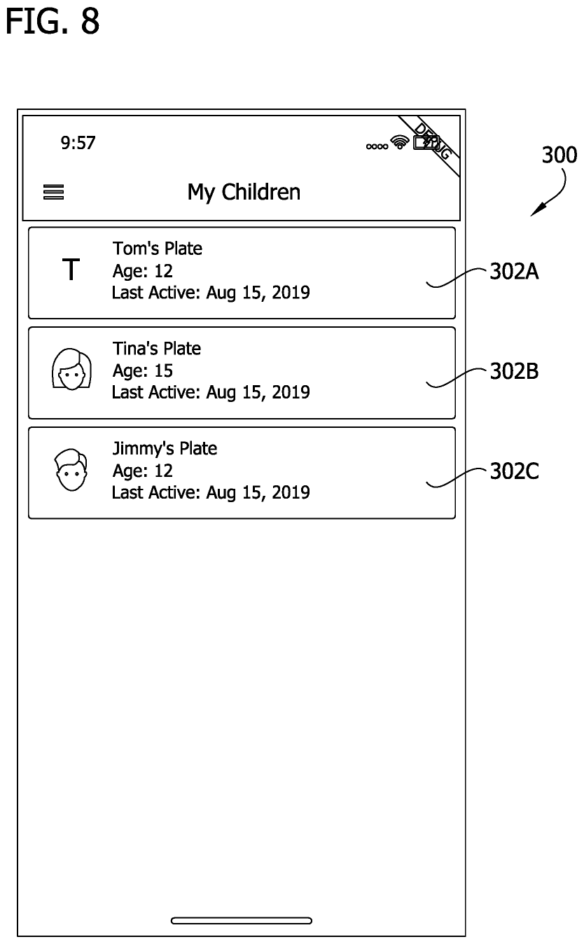

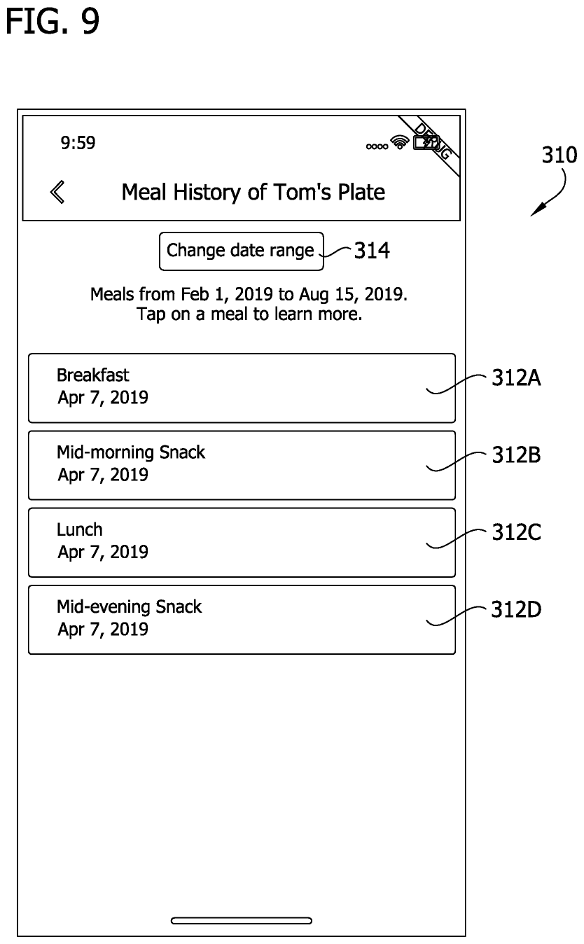

Referring to FIGS. 8-11, other devices (broadly, graphical user interfaces) can be communicatively coupled to the server to access the database and the information contained therein. For example, the server can be communicatively coupled to a mobile device (e.g., mobile phone, mobile computer, tablet, etc.) to permit an interested party, such as a parent, to access the information contained in the database. FIGS. 8-11 show screenshots of example views (graphical interfaces) that may be displayed on the graphical user interface, such as a mobile phone. These screenshots are of pages of an application (e.g., tray assembly monitoring application) operating on the graphical user interface, which has a touch sensitive screen. These screenshots demonstrate how a parent can monitor and track the food being consumed by their children using the tray assembly 100. FIG. 8 is a screenshot of a children home screen 300. The children home screen 300 has a plurality of individual child displays 302A-C listing the different children eating meals with the tray assembly 100. Each child display 302 is associated with one child of the parent. The child display 302 is also a button which can be actuated by the parent to view particular details about the food being consumed by the child associated with the child display. Pressing the child display 302 takes the parent to the meal history screen 310 for that child. For example, pressing on child display 302A takes the parent to the meal history screen 310 shown in FIG. 9.

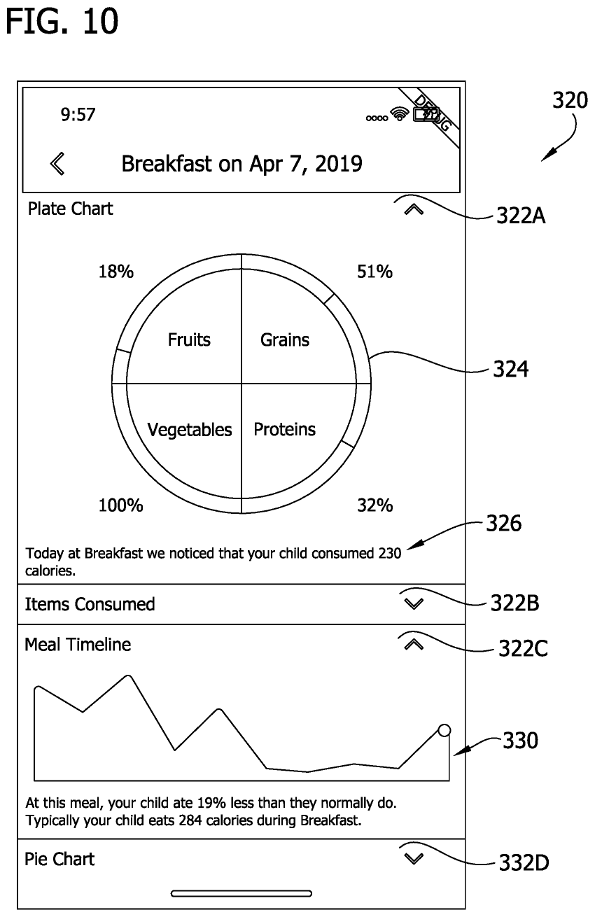

FIG. 9 is a screenshot of the meal history screen 310. The meal history screen 310 has a plurality of individual meal displays 312A-D listing the different meals eaten by the child. A date range button 314 may be used to change the date range of the meals shown in the meal history screen 310. The meal display 312 is also a button which can be actuated by the parent to view particular details about the meal consumed by the child associated with the meal display. Pressing the meal display 312 takes the parent to the meal detail screen 320 for that meal. For example, pressing on meal display 312A takes the parent to the meal detail screen 320 shown in FIGS. 9 and 10.

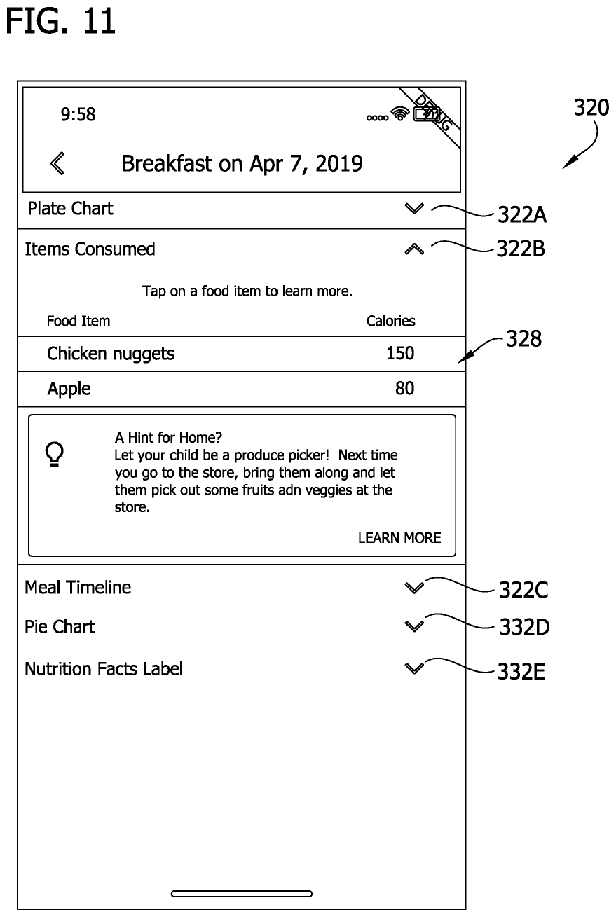

FIGS. 10 and 11 are screenshots of the meal detail screen 320. The meal detail screen 320 has a plurality of meal detail displays 322A-E listing different meal details the parent can view. In the illustrated embodiment, there are five meal detail displays 322A-E: Plate Chart, Items Consumed, Meal Timeline, Pie Chart, and Nutrition Facts Label. More or fewer meal detail displays 322A-E are within the scope of the present disclosure. Each meal detail display 322 is also a button which can be actuated by the parent to expand the meal detail screen 320 to view particular details of the meal shown by the selected meal detail display. For example, pressing on the meal detail display 322A labeled Plate Chart expands the meal detail screen 320 to display a food group chart 324 showing the categories of food (e.g., fruits, grains, vegetables, proteins, etc.) consumed by the child during that meal (e.g., breakfast) and the percentage of that category of food consumed relative to a target, expressed as a percentage. The target may be a recommended total amount of food consumed for that food category for that particular meal time (e.g., breakfast). The target may be based on USDA guidelines. A nutritional value 326 of the meal may also be displayed, such as the total calories consumed during the meal.

Pressing on the meal detail display 322B labeled Items Consumed expands the meal detail screen 320 to display a food item list 328 listing the types of food consumed and the calories consumed for each food type (FIG. 11). Pressing on the meal detail display 322C labeled Meal Timeline expands the meal detail screen 320 to display a timeline 330 generally showing how the food was consumed over the meal (FIG. 10). Pressing on the meal detail display 322D labeled Pie Chart expands the meal detail screen 320 to shown a daily intake pie chart (not shown) showing what meals were eaten that day and what percentage of the child's daily food intake came from each meal. Pressing on the meal detail display 322E labeled Nutrition Facts Label expands the meal detail screen 320 to display a nutrition facts chart (not shown) showing the nutritional information of the food consumed by the child for that day or meal. Of course, the information presented by these meal detail displays 322 is determined by referencing the weights of the different foods consumed and/or the nutritional information (e.g., calories) for the types of food consumed. All this information can be determined by the controller 150 and/or server and can be stored in the database. In addition, pressing on the meal detail display 322, while the display is open collapses that particular meal detail display. As needed, the parent can scroll through the meal detail screen to reach and select a desired meal detail display 322. For example, Nutrition Facts Label meal detail display 322E is hidden from view in FIG. 10, accordingly the parent would have to scroll upward to reveal and select this meal detail display. Additional detail displays may also be included in the meal detail screen 320. These additional detail display do not need to be food related. For example, one additional detail display could be a communication interface (e.g., API) to allow communication between parents using the mobile device and school administrators.

An exemplary method of the operation of the tray assembly 100 during a meal to monitor the food consumed from one or more dishes D will now be described. Initially, the tray assembly 100 is turned on and allowed to automatically connect to a wireless network (e.g., an initialization period). Once connected to the wireless network, the tray assembly 100 is ready to be used. To begin the meal, one or more dishes D are placed on the tray assembly 100 over the weight assembly 120. The weight assemblies 120 record the weight of each dish D as they are placed on the tray assembly 100 (e.g., measure the weight of each dish at a first time or at the beginning of the meal). The weight assemblies 120 then continue to measure the weight of each dish D and any food (including no food) contained therein during the meal. This may be done continuously or at predetermined intervals. In other words, the contents of each dish are accurately measured through the duration of the meal (e.g., a second time). This ensures the detailed caloric and nutritional intake of the user can be tracked and later viewed, as described herein.

At the end of the meal, the weight assemblies 120 record the weight of each dish D and any food contained therein. The end of the meal may be signaled by turning the tray assembly 100 off, with the last recorded weight for each weight assembly being the weight of the dishes D at the end of the meal (e.g., measuring the weight of each dish and any food contained therein at a second time or at the end of the meal). Alternatively, the controller 150 can include a button (not shown) on the housing 102 which can be actuated by a child to signal the start and/or end of a meal. Broadly speaking and in its simplest form, the amount of food consumed is determined by taking the difference between the weight taken at a first time during the meal and at a second time, after the first time, during the meal. In particular, the amount of food consumed may be determined by taking the difference between multiple periods of times (e.g., the difference between first and second times, third and fourth times, fifth and sixth time, etc.). There periods of times may be defined by local maximums (e.g., initial time) and minimums (e.g., later time), as discussed above in reference to FIG. 4. In other words, a difference between a plurality of local maximum and minimum weights taken during the meal is indicative of the amount of food consumed during the meal form the one or more dishes, with the first time being an exemplary one local maximum and the second time being an exemplary one local minimum. In addition, the weight of the dish D is analyzed over time to determine if any events occurred that should reduce or increase the amount of food consumed (e.g., the weight at multiple times are analyzed). For example, by analyzing the weight versus time for the dishes D, it can be determined that at least a portion of the food in one or more of the dishes D was not consumed. This can be based on a sudden decrease in the weight of the one or more dishes, as described above. Similarly, it can be determined that additional food has been added to one or more of the dishes D based on a sudden increase in the weight of one or more of the dishes, as described above. Once the amount of food consumed has been determined, this amount can then be used to determine other information, such as nutritional values for the meal as described herein. This information can then be displayed to other interested parties such as parents and/or school administrators.

Example methods and systems are described herein and illustrated in the accompanying drawings. The written description uses examples to disclose aspects of the disclosure and also to enable a person skilled in the art to practice the aspects, including making or using the above-described devices, assemblies, and/or systems and executing or performing the above-described operations.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. Furthermore, references to an “embodiment” or “example” of the present disclosure are not intended to be interpreted as excluding the existence of additional embodiments or examples that also incorporate the recited features. The terms “comprising,” “including,” and “having” are intended to be inclusive and mean that there may be additional elements other than the listed elements. The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

While aspects of the disclosure have been described in terms of various examples with their associated operations, a person skilled in the art would appreciate that a combination of operations from any number of different examples is also within the scope of the aspects of the disclosure.

What is claimed is:

1. A tray assembly for monitoring food consumed from one or more dishes supported by the tray assembly, the tray assembly comprising:
a housing defining an interior and having an upper surface for supporting the one or more dishes;
one or more weight assemblies disposed in the interior of the housing, each weight assembly including at least one weight sensor configured to detect a weight of one dish and any of the food contained therein of the one or more dishes and to provide a weight signal corresponding to the detected weight of the one dish;
wherein a difference between the weight taken at a first time during a meal and at a second time, after the first time, during the meal is indicative of an amount of food consumed during the meal from the one or more dishes.

2. The tray assembly of claim 1, wherein a difference between a plurality of local maximum and minimum weights taken during the meal is indicative of the amount of food consumed during the meal from the one or more dishes, wherein the weight taken at the first time is one local maximum weight of the plurality of local maximum weights and the weight taken at the second time is one local minimum weight of the plurality of local minimum weights.

3. The tray assembly of claim 1, the amount of food consumed during the meal is determined using a time-series algorithm.

4. The tray assembly of claim 1, wherein the amount of food consumed during the meal is further determined by excluding a difference between the weight taken at a third time during the meal and at a fourth time, after the third time, during the meal when the time difference between third and fourth times is two seconds or less.

5. The tray assembly of claim 1, further comprising a controller disposed in the interior of the housing and communicatively coupled to the one or more weight assemblies, the controller configured to receive the weight signal from the one or more weight assemblies.

6. The tray assembly of claim 5, wherein the controller is configured to determine, based on the weight signal, the amount of food consumed by taking the difference between the weight taken at the first time during the meal and the second time during the meal.

7. The tray assembly of claim 5, further comprising a server communicatively coupled to the controller, the controller configured to send a controller signal to the server corresponding to the detected weights of the one or more dishes, the server configured to determine, based on the controller signal, the amount of food consumed by taking the difference between the weight taken at the first time during the meal and at the second time during the meal.

8. The tray assembly of claim 7, wherein the controller is configured to automatically connect to a wireless network to communicatively couple the controller and server.

9. The tray assembly of claim 8, wherein the server is configured to determine the nutritional value of the food consumed based on referencing the amount of food consumed with the nutritional value of the food consumed.

10. The tray assembly of claim 1, wherein the housing includes a flexible mat defining the upper surface of the housing.

11. The tray assembly of claim 10, wherein the flexible mat is selectively removable from the rest of the housing.

12. The tray assembly of claim 10, wherein the one or more weight assemblies support at least a portion of the flexible mat, the flexible mat configured to transfer the weight of the one or more dishes to the one or more weight assemblies.

13. The tray assembly of claim 1, wherein each weight assembly includes a lower plate and an upper plate, the at least one weight sensor disposed between the upper and lower plates

14. The tray assembly of claim 1, wherein each weight assembly includes a dish locator configured to position one dish of the one or more dishes over said weight assembly when said one dish is supported by the tray assembly.

15. The tray assembly of claim 14, therein the dish locator of each weight assembly is one or more magnets, the one or more magnets configured to interact with a dish magnet on said one dish to position said one dish over the weight assembly.

16. The tray assembly of claim 15, further comprising the one or more dishes.

17. A method for monitoring food consumed from one or more dishes, the method comprising:
measuring a weight of each dish and food contained therein at a first time during the meal using a tray assembly;
measuring a weight of each dish and any of the food contained therein at a second time during the meal using the tray assembly; and
determining the amount of food consumed by taking the difference between the weight taken at the first and second times.

18. The method of claim 17, further comprising continuously measuring the weight of each dish during the meal.

19. The method of claim 18, further comprising determining at least a portion of the food in one or more of the dishes has not been consumed based on a sudden decrease in the weight of the one or more dishes.

20. The method of claim 19, further comprising determining additional food has been added to one or more of the dishes based on a sudden increase in the weight of the one or more dishes.
